(19) United States
(12) Patent Application Publication
(10) Pub. No.: US 2009/0299101 A1
(43) Pub. Date: Dec. 3, 2009

(54) Transition Metal Cluster Catalyst

(76) Inventors: Yasuhiro Uozumi, Okaxaki-shi (JP); Yoichi Yamada, Okaxaki-shi (JP)

Correspondence Address: GARY C. COHN, PLLC, P. O. Box 313, Huntingdon Valley, PA, 19006, US

(21) Appl. No.: 12/097,862

(22) PCT Filed: Oct. 11, 2006
(86) PCT No.: PCT/JP2006/320259

§ 371 Date: Jun. 17, 2008

(30) Foreign Application Priority Data
Dec. 19, 2005 (JP) 2005-365125

(51) Int. Cl. C07C 45/61; C08F 8/42

(52) U.S. Cl. 568/315; 525/371; 568/386

(57) ABSTRACT

The present invention provides a catalyst, which has enough catalytic activity as a transition metal particle catalyst including platinum family and the like, is easily separable from products, is reusable and is easily prepared. To prepare the transition metal cluster catalyst of the present invention, an insoluble complex is prepared by forming a complex between a polymer with nitrogen-containing group, such as pyridinium and ammonium group in the principal chain, and a later transition metal compound; and then reducing the complex with a reductant. The transition metal forms clusters, which are stabilized by the polymers. Namely, the present invention is a transition metal cluster catalyst, wherein transition metal clusters are supported by a polymer, which is obtained by reduction reaction of a complex of a transition metal and a polymer with nitrogen-containing group. The transition metal cluster catalyst of the present invention is an extremely useful catalyst for oxidation, reduction, cross-coupling, Heck reaction, alkylation reaction and the like.

### FIELD OF THE INVENTION

The present invention relates to a transition metal cluster catalyst and usage thereof in various reactions.

### PRIOR ART

Recently, palladium and platinum particles have been used in organic synthetic reactions. Since conventional reactions using metal nano-particles are homogenous systems and consequently separation of metals from products is difficult, metals remain in products to remarkably increase load in environment, which is a big problem. Furthermore, it is another problem that, even if metals are recovered, the metal catalyst becomes wastes eventually.

Therefore, it is required to develop such catalysts as those without having the above problems, i.e. catalysts effective without using organic solvents from a viewpoint of environment pollution and catalysts easily recoverable and reusable.

To resolve the above problems, it has been examined to subjecting an insoluble carrier to support metal particles. For example, the present inventors developed a polymeric resin catalyst containing palladium particles and performed oxidation of alcohols, reduction of alkenes and dechlorination reaction of allyl chloride (Reference 1).

Reference 1: WO2002/072644

### Problems to be Solved by the Invention

The polymeric resin catalyst containing palladium and platinum particles developed by the present inventors (Reference 1, Japanese Patent Application No. 2005-064911, Japanese Patent Application No. 2005-064913 etc.) is an excellent catalyst functional in water, recoverable and reusable. However, since the polymeric resin using is a modified resin, the catalyst is expensive, and also the catalyst needs a resin-supporting ligand. Then, the objective of the present invention is to develop a simple and inexpensive catalyst using a chain polymer without need of using a resin and a ligand, which is effectively functional in the absence of organic solvents.

Namely, the objective of the present invention is to provide a catalyst, which has enough catalytic activity as a transition metal particle catalyst including platinum family and the like, is easily separable from products, is reusable and is easily prepared.

### Means to Solve the Problems

To prepare the transition metal cluster catalyst of the present invention, an insoluble complex is prepared by forming a complex between a polymer with nitrogen-containing group, such as pyridinium and ammonium group in the principal chain, and a later transition metal compound; and then reducing the complex with a reductant. It is believed that during the above process, the complex becomes unstable and is destroyed, then the metal fine particles are incorporated into the polymer. In other words, a chain polymer becomes a matrix, and a transition metal forms clusters and is incorporated into the insoluble complex. In the product, the fine metal clusters bridge polymers and are in turn stabilized by the chain polymers. Therefore, a stable and reusable metal cluster catalyst is generated.

Therefore, the present invention is a transition metal cluster catalyst, wherein transition metal clusters are supported by a polymer, which is obtained by reduction reaction of a complex of a transition metal and a polymer, wherein the complex is represented by a general formula (1):

(—NR¹R²—R⁵—NR³R⁴—R⁶—)ₘM¹ₙ

wherein R¹, R², R³ and R⁴ represent independently an aryl group or an alkyl group, and NR¹R² and NR³R⁴ may form a pyridine ring, an acridine ring or a quinoline ring that may have substituent(s); R⁵ represents an arylene group, an alkylene group, or mixture of these groups that may have substituent(s); R⁶ represents a covalent bond or an alkylene group; M¹ represents a transition metal salt; m represents a number corresponding to molecular weight of the polymer; and n represents a number satisfying that m/n is from 1 to 10.

### BRIEF DESCRIPTION OF THE DRAWINGS

FIG. 1 shows a photograph of the field emission scanning electron microscope of the catalyst.

FIG. 2 shows a photograph of the field emission scanning electron microscope of the catalyst.

FIG. 3 shows the EDS (electron dispersion x-ray analysis) spectrum of the catalyst observed by the field emission scanning electron microscope.

### EFFECT OF THE PRESENT INVENTION

Organic solvents have been used for organic synthesis because of its prominent solubilizing agent for an organic compound. However, recently the use is severely restricted because of its effect as an environmental pollutant. Consequently, a reaction in the absence of an organic solvent or in the presence of water as a solvent is interfered because of hard solubility or insolubility of a common organic compound. However, the catalyst of the present invention is capable of effectively catalyzing various reactions in the presence of water as a solvent. Furthermore, conventional transition metal catalyst needs to be used in the absence of oxygen or in the presence of inert gas, whereas the catalyst of the present invention has the advantage in that it can be used in the atmosphere.

The transition metal cluster catalyst of the present invention is an extremely useful catalyst for oxidation, reduction, cross-coupling, Heck reaction and alkylation reaction. Particularly, since alkylation process using the transition metal cluster catalyst of the present invention needs not to use highly toxic alkyl halide as a nucleophilic reagent and may use a primary alcohol, the process is capable of realizing reaction system without generating halogens and is an excellent “green chemistry”-oriented process.

### DETAILED DESCRIPTION OF THE INVENTION

The transition metal cluster catalyst of the present invention is obtained by reduction reaction of a complex comprising a transition metal salt and a polymer, wherein the complex is represented by the general formula (1):

C1: (—NR¹R²—R⁵—NR³R⁴—R⁶—)ₘM¹ₙ

wherein R¹, R², R³ and R⁴ represent independently an aryl group or an alkyl group, and NR¹R² and NR³R⁴ may form a pyridine ring, an acridine ring or a quinoline ring, and preferably a pyridine ring, which may have substituent(s). The aryl group is preferably phenyl group and the carbon number of alkyl group is preferably equal to or less than 20. The substituent is preferably an aryl group or an alkyl group, and the aryl group is preferably phenyl group. The carbon number of alkyl group is preferably equal to or less than 4.

R⁵ represents an arylene group, an alkylene group, or mixture of these groups, which may have substituent(s). The alkylene group has preferably carbon number between 1 and 20, is more preferably linear, and is even more preferably an alkylene group represented by —(CH₂)ₙ—, wherein the carbon number (n) is preferably equal to or less than 10 and more preferably between 1 and 6. The arylene group is preferably a phenylene group or a naphthylene group. The substituent is preferably an aryl group or an alkyl group, the aryl group is preferably a phenyl group, and the alkyl group has preferably carbon number equal to or less than 4.

R⁶ represents a covalent bond or an alkylene group. The alkylene group has preferably carbon number between 1 and 20, is preferably linear, and is more preferably an alkylene group represented by —(CH₂)ₙ—, wherein the carbon number (n) is preferably equal to or less than 10 and more preferably between 4 and 6.

M¹ represents a transition metal salt and is represented by MXₜ. M is a transition metal, preferably a later transition metal (iron group and platinum group), more preferably palladium, nickel, platinum, cobalt, rhodium or iridium, even more preferably palladium or platinum.

X includes a halogen atom, carboxylate group (—OCOR⁷, wherein R⁷ is not restricted, but is preferably a hydrocarbon group, more preferably an alkyl group or an aryl group), a carbonate group (CO³⁻), a phosphate group (PO₄³⁻), a sulphate group (SO₄²⁻), and a nitrate group (NO³⁻). t is an integer leading MXₜ to divalent anion.

m represents a number corresponding to molecular weight of the polymer. The molecular weight of the polymer depends on synthetic conditions, but is generally between about 5,000 and 1,000,000. n represents a number satisfying the ratio m/n is between 1 and 10. The ratio m/n is preferably selected so that the charge number of the quaternary ammonium is stoichiometrically balanced with that of the transition metal salt.

The preferable example of the complex includes a compound represented by the general formula (2):

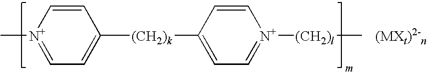

wherein k is a number corresponding to R⁵, l is a number corresponding to R⁶, and m, n, M, X and t are as defined above, and the pyridine ring may contain substituent(s), or represented by the general formula (3):

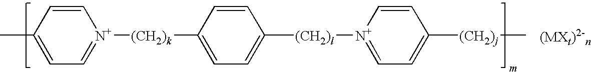

wherein k and l represent the number corresponding to R⁵, j is a number corresponding to R⁶, and m, n, M, X and t are as defined above, and the pyridine ring and the benzene ring may contain substituent(s).

The complex can be obtained, for example, by a reaction between a tertiary amine compound and a halogen compound to synthesize a polymer containing quaternary ammonium, and then by a reaction between the polymer and a transition metal salt.

The tertiary amine is represented by the following formula (4):

NR¹R²—R⁵—NR³R⁴,

wherein R¹, R², R³, R⁴, R⁵ are as defined above.

The halogen compound is represented by the following formula (5):

X¹—R⁶—X²,

wherein X¹ and X² represent independently a halogen atom, preferably a chlorine atom or a bromide atom, and R⁶ is as defined above.

These tertiary amine compounds are reacted with the halogen compound. A highly polar solvent is preferably used as a solvent and includes acetonitril, acetone, dimethylformamide, dimethylacetamide, t-butyl alcohol and the like.

The concentration of the reactants is between about 0.01 and 1 M, and preferably about 0.25 M.

The atmosphere of the reaction is any of air, nitrogen and argon.

The reaction temperature is selected between 0° C. and the reflux temperature of the solvent, and is preferably about 82° C. The reaction time is between about 1 and 144 hr, and preferably about 24 hr.

As a result of the reaction, a polymer containing a quaternary ammonium represented by the following formula (6) is obtained:

(—NR¹R²—R⁵—NR³R⁴—R⁶)ₘ,

wherein R1, R2, R3, R4, R5 are as defined above and the molecular weight is generally between about 5,000 and 1,000,000 under general reaction conditions.

Then the polymer is reacted with the above transition metal salt.

A highly polar solvent is preferably used as a solvent and includes water, methanol, ethanol, propanol, 2-propanol, t-butanol, chloroform and the like. Especially, water is preferably used.

The concentrations of the reactants are between about 0.001 and 0.1 M, and preferably about 0.01 M.

The atmosphere of the reaction is any of air, nitrogen and argon.

The reaction temperature is selected between −78° C. and 100° C., and is preferably around at room temperature. The reaction time is between about 1 sec and 7 days, and preferably about 1 hr.

After the reaction, the complex of formula (1) comprising the transition metal and the polymer is obtained as an insoluble product. The present complex is insoluble to water and the above organic solvent, and is able to recover and reuse. The recovery method includes filtration, centrifugation, recovery of the supernatant, and the like.

Then the complex is subjected to reduction reaction.

The reductant used to the reduction reaction includes a metal hydride reagent, a metal or ammonium salt of formic acid, a primary or secondary alcohol, and hydrogen, and a metal hydride reagent is preferably used among them.

A metal hydride reagent includes an alkali metal, alkali earth metal, or an ammonium salt of aluminum metal family (boron, aluminum and the like) hydride, and includes specifically NaBH₄, LiBH₄, LiAlH₄ and the like.

A metal or ammonium salt of formic acid includes preferably an alkali metal, alkali earth metal, or an ammonium salt of formic acid, and precisely formic acid and a metal or ammonium salt thereof such as formic acid, ammonium formate and sodium formate. A primary or secondary alcohol includes methanol, ethanol, propanol, 2-propanol, butanol, benzilalcohol, and the like.

The reduction reaction can be performed in the presence or absence of a solvent. For the reaction in the presence of a solvent, the solvent includes water, alcohols such as methanol, ethanol, 2-propanol, butanol, benzilalcohol, and preferably ethanol, tetrahydrofrane, methyltetrahydrofrane, tetrahydropyrane, and ethers such as diethylether, diisopropylether. The reaction mixture is added with a reductant at the temperature less than the melting temperature of a solvent, wherein the temperature is between 0 and 100° C. for water, between −78° C. and 150° C. for alcohols, and preferably 25° C.; is stirred for period between 0.1 sec and 72 hr, preferably about 6 hr, at the temperature between −78° C. and 150° C., preferably at 25° C.; and generates the desired cluster catalyst.

The transition metal cluster catalyst is stabilized in a state, wherein transition metal clusters with diameter between about 1 and 5 nm are supported by the polymers.

The catalyst of the present invention is effectively functional in oxidation reaction, reduction reaction, homo-coupling reaction, cross-coupling reaction, Heck reaction, alkylation reaction or the like, and particularly for α-alkylation reaction.

In the α-Alkylation reaction, any kinds of ketones containing α-hydrogen can be used as a substrate, and any kinds of primary alcohols can be used as a reagent.

An alkylated product at α-site of substrate ketone is produced by the reaction using alkali, alkali earth metal base, amines as a base, at the temperature between −78° C. and 200° C., in the absence of a solvent, in the presence of a highly polar solvent such as water, alcohol, dimethylformamide and the like or in the presence of a nonpolar solvent such as toluene, ether, hydrocarbon and the like.

The α-alkylation reaction is represented by the following reaction formula (7):

R⁸—CO—CH(R⁹)+HO—R¹⁰→R¹—CO—C(R⁹)₂—R¹⁰,

wherein R⁸, R⁹ and R¹⁰ are not restricted but each may represent hydrocarbon group, and R⁹ is preferably a hydrogen atom.

In reduction reaction, for example, by allowing a compound with double bond or triple bond such as alkene or alkine to react with hydrogen, formic acid or salts thereof in the presence of alcohol at the temperature between −78° C. and 150° C., a corresponding alkane can be produced.

In oxidation reaction, for example, by allowing alcohols to react with an oxidant such as air, oxygen, hydrogen peroxide, t-butylhydroperoxide, dimethylsilylperoxide, or the like at the temperature between −78° C. and 150° C., a corresponding ketone, aldehyde or carboxylic acid can be produced.

In coupling reaction, for example, by allowing aryl halides, alkenyl halides or alkane halides to react in the presence of an organic metal reagent (organic boron, organic aluminum, organic zinc or organic zirconium) at the temperature between −78° C. and 200° C., a corresponding coupling compound can be produced.

In Heck reaction, for example, by allowing aryl halides, alkenyl halides or alkane halides to react with alkenes at the temperature between −78° C. and 200° C., a corresponding arylalkene, dialkene, or alkene can be produced.

The following Examples illustrate the present invention, but are not intended to limit the scope of the present invention.

### Example 1

4,4′-bipyridine (1.56 g; 10 mmol: Tokyo Chemical Industry, Co., Ltd.) and 1,4-bis(bromomethyl)toluene (2.64 g; 10 mmol: Aldrich) are dissolved in acetonitrile (50 mL) and water (50 mL) and the solution was stirred at 100° C. for 24 hr. After the reactant was cooled to a room temperature, it was subjected to an evaporator for removal of the solvent, was washed with chloroform (200 mL), acetone (200 mL) and chloroform (200 mL), was dried under reduced pressure. As the result, poly{(1,4-bipyridil)-co-[1,4-bis(bromomethyl)benzene]}(the following compound 1) was obtained (4.0 g, yield>99%). The analytical result is shown as follows:

CP-MAS ¹³C NMR (232 MHz; solid) 148.1, 145.2, 135.1, 133.1, 127.3, 60.8; calcd. for C₁₈H₁₆Br₂N₂.2H₂O: C, 47.39%; H, 4.42%; N, 6.14%. found: C, 47.98%; H, 4.24%; N, 6.27%.

The obtained aqueous solution (100 mL) dissolving palladium chloride (Furuya Metal Co., Ltd.)(4 mmol) and sodium chloride (80 mmol) was mixed with the aqueous solution (100 mL) of the obtained poly{(1,4-bipyridil)-co-[1,4-bis(bromomethyl)benzene]}(4 mmol; 1.68 g) at 25° C. The mixture generated precipitation. After the mixture was stirred for further 1 hr, the precipitation was filtrated, washed with water, and dried. As the result, an insoluble product was obtained (the following compound 2)(1.77 g; yield 87%). The analytical result is shown as follows:

CP-MAS ¹³C NMR (232 MHz; solid) δ 148.9, 146.8, 135.0, 131, 1, 128.0, 64.4; IR (ATR) v 3471, 3117, 3055, 2920, 2851, 1636, 1611, 1436, 1421, 809, 768 cm⁻¹; Anal. calcd. for C₁₈H₁₆Br₂Cl₂N₂Pd.3H₂O: C, 33.18%; H, 3.40%; N, 4.30%. found: C, 31.91%; H, 2.66%; N, 4.33%.

The obtained insoluble product (1.77 mmol; 900 mg) was dispersed in ethanol (75 mL), was slowly mixed with sodium boron hydride (Wako Pure Chemical Industry Lyd., 11.9 mmol) dispersed in ethanol (75 mL) at 25° C., and the mixture was changed to a black dispersion solution. The solution was stirred for further 6 hr, and the precipitates were filtrated, washed with water and dried. As the result, an insoluble product (the following formula, the catalyst of compound 3)(630 g; yield 81%) was obtained. The analytical result is shown as follows:

CP-MAS ¹³C NMR (232 MHz; solid) δ 130.9, 128.8, 63.9, 56.8, 52.8, 42.1, 31.5, 15.5; IR (ATR) v 3471, 3117, 3054, 2920, 2851, 1636, 1436, 1236, 1090, 891 cm⁻¹; Anal. calcd. for C₁₈H₁₆Br₂N₂Pd.3H₂O: C, 37.24%; H, 3.82%; N, 4.82%. found: C, 37.51%; H, 3.72%; N, 5.08%.

The reaction of the present Example is shown by the following reaction formula (8):

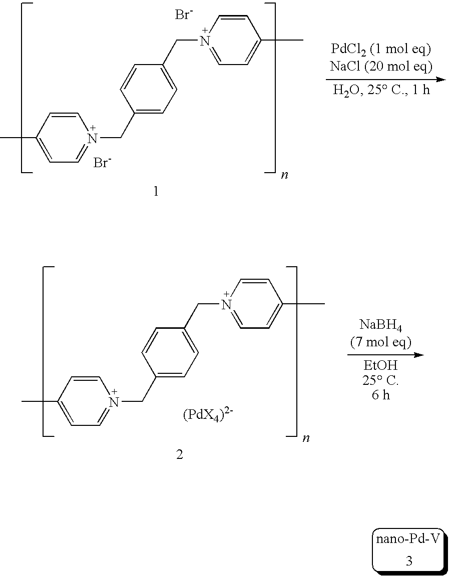

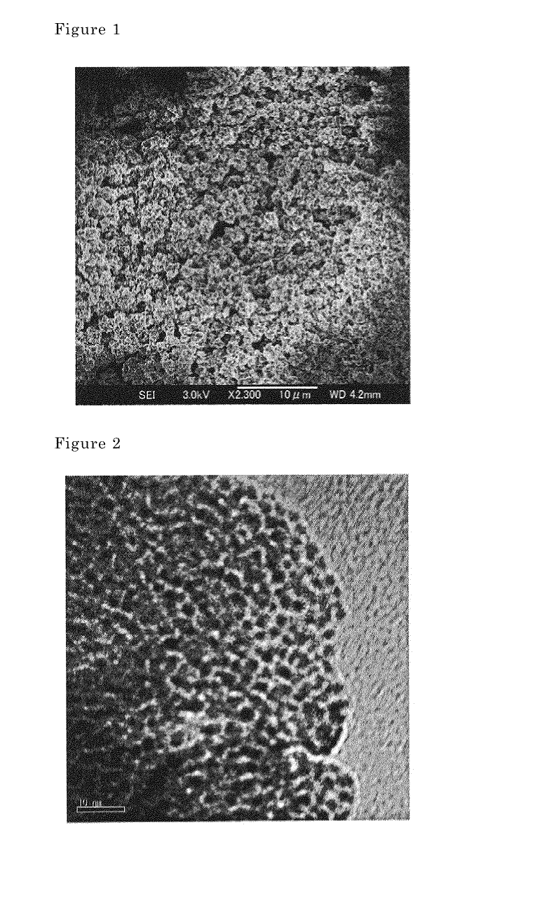

The obtained catalyst was examined by a field emission scanning electron microscope (JEOL Ltd., JSM-6700, Voltage 5 kV, Magnification ×2300). The photograph is shown in FIG. 1. FIG. 1 shows a micrometer-scale configuration of the catalyst and exemplifies porosity with size of about 1 μm.

Measurement by field emission transmission electron microscopy (JEOL Ltd., JEM-2100F, Voltage 200 kV, Magnification ×250000) was performed. The photograph is shown in FIG. 2. FIG. 2 shows that the black colored region is palladium, and a polymer is present at the border. It was observed that palladium clusters were dispersed on aggregated polymers.

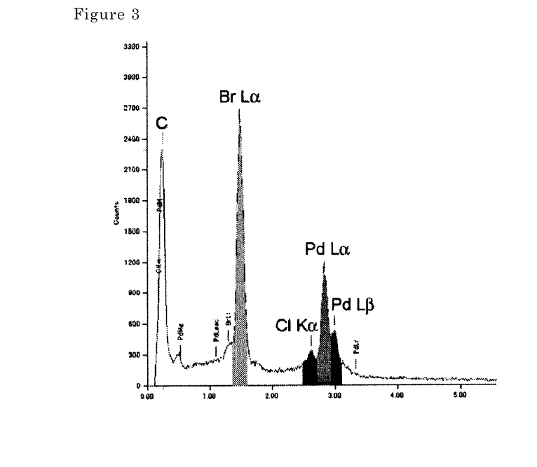

Energy dispersive X-ray analysis (EDS) was performed by field emission scanning electron microscopy (JSM-6700). The result is shown in FIG. 3. FIG. 3 exemplified that palladium clusters were generated, bromide anions were present on the polymer as anions, and a small amount of chlorides were contained.

The Pd clusters generated in the present Example have diameter of about 2 nm according to the observation by field emission transmission electron microscopy, have neutral charge according to the reaction mechanism of generating zero valent neutral clusters by reduction of divalent palladium, and are carried by the polymer.

### Example 2

The catalyst obtained in Example 1 (10 mg), barium hydroxide monohydrate (63 mg), water (42 μL), 2-octanone (0.334 mmol), and 1-octanol (0.668 mmol) were stirred at 100° C. for 24 hr under air atmosphere, were added with ethyl acetate after cooling, and were centrifuged (4000 rpm, 5 min) to provide supernatant. The supernatant was concentrated, was purified by a column chromatography, and provided 7-hexadecanone at the yield of 83%. The catalyst recovered by the centrifugation was washed with water, was dried for 12 hr under 5 Pascal, and was reused for the same reaction to provide 7-hexadecanone at the yield of 90%. Further the same procedure provided 7-hexadecanone at the yield of 91%.

¹H NMR (CDCl₃) 2.38 (t., J=7.6 Hz, 4H), 1.54-1.59 (m, 4H), 1.26-1.31 (m, 18H), 0.88 (t, J=6.7 Hz)

The reaction of the present Example is shown by the following reaction formula (9)

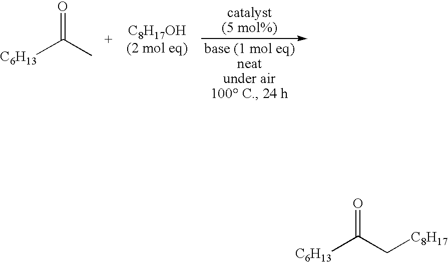

### Example 3

The catalyst obtained in Example 1 (10 mg), barium hydroxide monohydrate (63 mg), water (42 μL), 2-octanone (0.334 mmol), and 1-decanol (0.668 mmol) were stirred at 100° C. for 24 hr under air atmosphere, were added with ethyl acetate after cooling, and were centrifuged (4000 rpm, 5 min) to provide supernatant. The supernatant was concentrated, was purified by a column chromatography, and provided 7-octadecanone at the yield of 84%.

¹H NMR (CDCl₃) 2.31 (t, J=8 Hz, 4H), 1.47-1.51 (m, 4H), 1.15-1.31 (m, 12H), 0.81 (t, J=7 Hz, 6H)

The reaction of the present Example is shown by the following reaction formula (10)

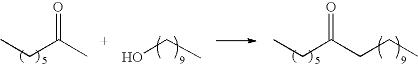

### Example 4

The catalyst obtained in Example 1 (10 mg), barium hydroxide monohydrate (63 mg), water (42 μL), 2-octanone (0.334 mmol), and benzyl alcohol (0.668 mmol) were stirred at 100° C. for 24 hr under air atmosphere, were added with ethyl acetate after cooling, and were centrifuged (4000 rpm, 5 min) to provide supernatant. The supernatant was concentrated, was purified by a column chromatography, and provided 1-phenyl-3-nonanone at the yield of 91%.

¹H NMR (CDCl₃) 7.25-7.28 (m, 2H), 7.17-7.19 (m, 3H), 2.89 (t, J=7.3 Hz, 2H), 2.72 (t, J=7.3 Hz, 2H), 2.37 (t, J=7.3 Hz, 2H), −1.52-1.56 (m, 2H), 1.24-1.30 (m, 6H), 0.87 (t, 6.7 Hz, 3H)

The reaction of the present Example is shown by the following reaction formula (11):

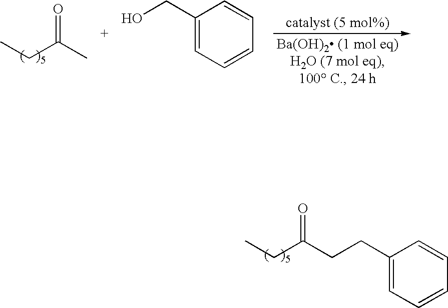

1. A transition metal cluster catalyst, wherein transition metal clusters are supported by a polymer, which is obtained by reduction reaction of a complex of a transition metal and a polymer, wherein the complex is represented by a general formula (1):
(—NR¹R²—R⁵—NR³R⁴—R⁶—)ₘM¹ₙ
wherein R¹, R², R³ and R⁴ represent independently an aryl group or an alkyl group, and NR¹R² and NR³R⁴ may form a pyridine ring, an acridine ring or a quinoline ring that may have substituent(s); R⁵ represents an arylene group, an alkylene group, or mixture of these groups that may have substituent(s); R⁶ represents a covalent bond or an alkylene group;
M¹ represents a transition metal salt; m represents a number corresponding to molecular weight of the polymer; and n represents a number satisfying that m/n is from 1 to 10.

2. The catalyst of claim 1, wherein the transition metal is palladium, nickel, platinum, cobalt, rhodium, or iridium.

3. The catalyst of claim 1, wherein the transition metal salt is represented by MXₜ
wherein M represents a transition metal, X represents a halogen atom, a carboxylate group, a carbonate group, a phosphate group, a sulfate group or a nitrate group, and t represents an integer so that MXₜ is a divalent anion.

4. The catalyst of claim 3, wherein the complex is represented by a general formula (2): 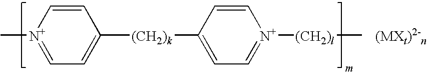
wherein k is a number corresponding to R⁵, 1 is a number corresponding to R⁶, and m, n, M, X and t are as defined above, and the pyridine ring may contain substituent(s),
or represented by a general formula (3): 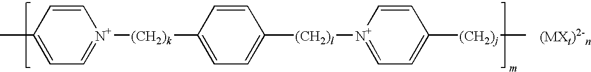
wherein k and l represent the number corresponding to R⁵, j is a number corresponding to R⁶, and m, n, M, X and t are as defined above, and the pyridine ring and the benzene ring may contain substituent(s).

5. A process comprising conducting an oxidation reaction, reduction reaction, coupling reaction, Heck reaction, or alkylation reaction in the presence of a catalyst of claim 1.

6. The process of claim 5 wherein the reaction is an α-alkylation reaction for producing a ketone whose α-site is alkylated by using a ketone having α-hydrogen as a substrate and a primary alcohol as a reagent.

7. The catalyst of claim 2, wherein the transition metal salt is represented by MXₜ wherein M represents a transition metal, X represents a halogen atom, a carboxylate group, a carbonate group, a phosphate group, a sulfate group or a nitrate group, and t represents an integer so that MXₜ is a divalent anion.

8. The catalyst of claim 7, wherein the complex is represented by a general formula (2): 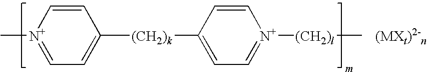
wherein k is a number corresponding to R⁵, 1 is a number corresponding to R⁶, and m, n, M, X and t are as defined above, and the pyridine ring may contain substituent(s),
or represented by a general formula (3): 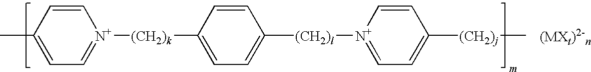
wherein k and l represent the number corresponding to R⁵, j is a number corresponding to R⁶, and m, n, M, X and t are as defined above, and the pyridine ring and the benzene ring may contain substituent(s).

9. A process comprising conducting an oxidation reaction, reduction reaction, coupling reaction, Heck reaction, or alkylation reaction in the presence of a catalyst of claim 2.

10. The process of claim 9 wherein the reaction is an α-alkylation reaction for producing a ketone whose α-site is alkylated by using a ketone having α-hydrogen as a substrate and a primary alcohol as a reagent.

11. A process comprising conducting an oxidation reaction, reduction reaction, coupling reaction, Heck reaction, or alkylation reaction in the presence of a catalyst of claim 3.

12. The process of claim 11 wherein the reaction is an α-alkylation reaction for producing a ketone whose α-site is alkylated by using a ketone having α-hydrogen as a substrate and a primary alcohol as a reagent.

13. A process comprising conducting an oxidation reaction, reduction reaction, coupling reaction, Heck reaction, or alkylation reaction in the presence of a catalyst of claim 4.

14. The process of claim 13 wherein the reaction is an α-alkylation reaction for producing a ketone whose α-site is alkylated by using a ketone having α-hydrogen as a substrate and a primary alcohol as a reagent.

15. A process comprising conducting an oxidation reaction, reduction reaction, coupling reaction, Heck reaction, or alkylation reaction in the presence of a catalyst of claim 7.

16. The process of claim 15 wherein the reaction is an α-alkylation reaction for producing a ketone whose α-site is alkylated by using a ketone having α-hydrogen as a substrate and a primary alcohol as a reagent.

17. A process comprising conducting an oxidation reaction, reduction reaction, coupling reaction, Heck reaction, or alkylation reaction in the presence of a catalyst of claim 8.

18. The process of claim 17 wherein the reaction is an α-alkylation reaction for producing a ketone whose α-site is alkylated by using a ketone having α-hydrogen as a substrate and a primary alcohol as a reagent.
